(19) United States
(12) Patent Application Publication
(10) Pub. No.: US 2017/0369755 A1
(43) Pub. Date: Dec. 28, 2017

(54) HYDROFLUOROOLEFINS AND METHODS FOR USING SAME

(71) Applicant: 3M INNOVATIVE PROPERTIES COMPANY, St. Paul, MN (US)

(72) Inventor: Zhongxing Zhang, Woodbury, MN (US)

(21) Appl. No.: 15/533,770

(22) PCT Filed: Dec. 1, 2015
(86) PCT No.: PCT/US15/63076

§ 371 Date: Jun. 7, 2017

Related U.S. Application Data
(60) Provisional application No. 62/088,770, filed on Dec. 8, 2014.

(51) Int. Cl. C09K 5/10; C07C 21/18; H01L 21/67; B23K 1/012

(52) U.S. Cl. CPC C09K 5/10; H01L 21/67109; C07C 21/18; B23K 1/012

(57) ABSTRACT

A composition that includes a hydrofluoroolefin represented by the following general formula (I): Rf—CH2CH—CHCH2-Rf (I). Rf is a perfluoroalkyl group having 6 carbon atoms, and the hydrofluoroolefin is a liquid at room temperature.

### FIELD

This disclosure relates to compositions, apparatuses, and methods that include hydrofluoroolefins.

### BACKGROUND

Various hydrofluoroolefins are described in, for example, U.S. Pat. App. Pub. 2014/0031442, U.S. Pat. App. Pub. 2013/0096218, and U.S. Pat. App. Pub. 2007/0096051.

### SUMMARY

In some embodiments, a composition that includes a hydrofluoroolefin is provided. The hydrofluoroolefin is represented by the following general formula (I):

Rf—CH2CH═CHCH2-Rf (I)

Rf is a perfluoroalkyl group having 6 carbon atoms, and the hydrofluoroolefin is a liquid at room temperature.

In some embodiments, a working fluid that includes the above-described hydrofluoroolefin is provided. The hydrofluoroolefin is present in the working fluid at an amount of at least 50% by weight based on the total weight of the working fluid. In some embodiments, an apparatus for heat transfer is provided. The apparatus includes a device, and a mechanism for transferring heat to or from the device. The mechanism includes a heat transfer fluid that includes the above described hydrofluoroolefin.

In some embodiments, a method of transferring heat is provided. The method includes providing a device, and transferring heat to or from the device using a heat transfer fluid that includes the above-described composition or working fluid. The above summary of the present disclosure is not intended to describe each embodiment of the present disclosure. The details of one or more embodiments of the disclosure are also set forth in the description below. Other features, objects, and advantages of the disclosure will be apparent from the description and from the claims.

### DETAILED DESCRIPTION

Presently, various fluids are used for heat transfer. The suitability of the heat transfer fluid depends upon the application process. For example, in some electronic applications, a heat-transfer fluid which is inert, has a high dielectric strength, low toxicity, good environmental properties, and good heat transfer properties over a wide temperature range is desirable.

Vapor phase soldering is a process application that requires heat transfer fluids which are especially suitable for the high temperature exposure. In such application, temperatures of between 170° C. and 250° C. are typically used with 200° C. being particularly useful for soldering applications using a lead based solder and 230° C. useful for the higher melting lead free solders. Currently, the heat transfer fluids used in this application are of the perfluoropolyether (PFPE) class. While many PFPEs have adequate thermal stability at the temperatures employed, they also possess the notable drawback of being environmentally persistent with extremely long atmospheric lifetimes which, in turn, gives rise to high global warming potentials (GWPs). As such, there is a need for new materials which possess the characteristics of the PFPEs that make them useful in vapor phase soldering as well as in other high temperature heat transfer applications (e.g., high dielectric strength, low electrical conductivity, chemical inertness, thermal stability and effective heat transfer, liquid over a wide temperature range, good heat-transfer properties over a wide range of temperatures), but which have a much shorter atmospheric lifetime and lower GWPs.

In this disclosure:

“device” refers to an object or contrivance which is heated, cooled, or maintained at a predetermined temperature;

“inert” refers to chemical compositions that are generally not chemically reactive under normal conditions of use;

“mechanism” refers to a system of parts or a mechanical appliance; and

“perfluoro-” (for example, in reference to a group or moiety, such as in the case of

“perfluoroalkylene” or “perfluoroalkylcarbonyl” or “perfluorinated”) means completely fluorinated such that, except as may be otherwise indicated, there are no carbon-bonded hydrogen atoms replaceable with fluorine;

“tertiary nitrogen” refers to a nitrogen atom with three substituents other than hydrogen; and

“terminal” refers to a moiety or chemical group that is at the end of a molecule or has only one group attached to it.

As used herein, the singular forms “a”, “an”, and “the” include plural referents unless the content clearly dictates otherwise. As used in this specification and the appended embodiments, the term “or” is generally employed in its sense including “and/or” unless the content clearly dictates otherwise.

As used herein, the recitation of numerical ranges by endpoints includes all numbers subsumed within that range (e.g. 1 to 5 includes 1, 1.5, 2, 2.75, 3, 3.8, 4, and 5).

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

In some embodiments, the present disclosure is directed to a hydfluoroolefin represented by the following general formula (I):

Rf—CH2CH═CHCH2-Rf (I)

where Rf is a perfluoroalkyl group having 6 carbon atoms. In some embodiments, the hydfluoroolefin may be represented by the following formula (II):

CF3CF2CF2C(CF3)2CH2CH═CHCH2C(CF3)2CF2CF2CF3 (II)

It is to be appreciated that the hydrofluoroolefins of the present disclosure may include the cis isomer, the trans isomer, or a mixture of the cis and trans isomers.

In some embodiments, the hydrofluoroolefins of the present disclosure may exhibit properties that render them particularly useful as heat transfer fluids for the electronics industry. For example, the hydfluoroolefins may be chemically inert (i.e., they do not easily react with base, acid, water, etc.), and may have high boiling points (up to 300° C.), low freezing points (they may be liquid at −40° C. or lower), low viscosity, high thermal stability, good thermal conductivity, adequate solvency in a range of potentially useful solvents, and low toxicity. The hydfluoroolefins may also, surprisingly, be liquid at room temperature (e.g., between 20 and 25° C.), as opposed to similar known hydfluoroolefins, which are solid at room temperature.

Hydrocarbon alkenes are known to react with hydroxyl radicals and ozone in the lower atmosphere at rates sufficient to lead to short atmospheric lifetimes (see Atkinson, R.; Arey, J., Chem Rev. 2003, 103 4605-4638). For example, ethene has an atmospheric lifetime by reaction with hydroxyl radicals and ozone of 1.4 days and 10 days, respectively. Propene has an atmospheric lifetime by reaction with hydroxyl radicals and ozone of 5.3 hours and 1.6 days, respectively. Both the cis and trans isomers of hydrofluoroolefins of the present disclosure were found to react at a very high rate with ozone in the gas phase. As a result, it is believed that these compounds have relatively short atmospheric lifetimes.

Furthermore, in some embodiments, the hydrofluoroolefins of the present disclosure may have a low environmental impact. In this regard, the hydfluoroolefins may have a global warming potential (GWP) of less 300, 200, 100 or even less than 10. As used herein, GWP is a relative measure of the warming potential of a compound based on the structure of the compound. The GWP of a compound, as defined by the Intergovernmental Panel on Climate Change (IPCC) in 1990 and updated in 2007, is calculated as the warming due to the release of 1 kilogram of a compound relative to the warming due to the release of 1 kilogram of CO2 over a specified integration time horizon (ITH).

$${GWP}_{i} ( {t}^{′} ) = \frac{{\int }_{0}^{ITH} {a}_{t} [ C ( t ) ] dt}{{\int }_{0}^{ITH} {a}_{CO 2} [ {C}_{CO 2} ( t ) ] dt} = \frac{{\int }_{0}^{ITH} {a}_{i} {C}_{oi} {e}^{- t / n} dt}{{\int }_{0}^{ITH} {a}_{CO 2} [ {C}_{CO 2} ( t ) ] dt}$$

In this equation aᵢ is the radiative forcing per unit mass increase of a compound in the atmosphere (the change in the flux of radiation through the atmosphere due to the IR absorbance of that compound), C is the atmospheric concentration of a compound, τ is the atmospheric lifetime of a compound, t is time, and i is the compound of interest. The commonly accepted ITH is 100 years representing a compromise between short-term effects (20 years) and longer-term effects (500 years or longer). The concentration of an organic compound, i, in the atmosphere is assumed to follow pseudo first order kinetics (i.e., exponential decay). The concentration of CO2 over that same time interval incorporates a more complex model for the exchange and removal of CO2 from the atmosphere (the Bern carbon cycle model).

In some embodiments, the above-described hydrofluoroolefins may be prepared by using halogenated butene such as, for example, 1,4-dibromobutene, 1-chloro,4-bromobutene, 1,4-dichlorobutene, 1,4-diiodobutene, or the mixture of these butenes as an alkylating agent. Addition of fluoride ion, F—, to a perfluoroolefin can form a fluorocarbanion which can be alkylated to form the desired product. In some embodiments, the fluoride ion sources may be metal salts of fluoride such as KF, CsF, AgF, or CuF, individually, or as a mixture thereof. Other halogen salt such as KBr, CsBr, AgBr, CuBr, KI, CsI, AgI, CuI can be used to promote the formation of the fluorocarbanion or assist the alkylation reaction. The perfluoroolefin can be one or a mixture of (trans)-1,1,1,2,3,4,5,5,5-nonafluoro-4-(trifluoromethyl)pent-2-ene, (cis)-1,1,1,2,3,4,5,5,5-nonafluoro-4-(trifluoromethyl)pent-2-ene or 1,1,1,3,4,4,5,5,5-nonafluoro-2-(trifluoromethyl)pent-2-ene. The amount of fluoride ion may be at least a stoichiometric amount, i.e., one mole of perfluoroolefin requires one mole or more of fluoride ion. A polar organic solvent may be used to dissolve sufficient amount of fluorocarbanion and alkylating agent in order for the reaction to occur. Many polar solvents such as acetonitrile, benzonitrile, N,N-dimethylformamide (DMF), bis(2-methoxyethyl) ether (diglyme), tetraethylene glycol dimethyl ether (tetraglyme), tetrahydrothiophene-1,1-dioxide (sulfolane), N-methyl-2-pyrrolidinone (NM2P), dimethyl sulfone can be used individually or as a mixture. In some embodiments, one or more catalysts may be employed. Suitable catalysts may include quaternary ammonium salt, phosphonium salt, and crown ethers, such as 18-crown-6, dibenzo-18-crown-6, diaza-18-crown-6,12-crown-4,15-crown-5, or combinations thereof.

In some embodiments, the present disclosure is further directed to working fluids that include the above-described hydrofluoroolefins as a major component. For example, the working fluids may include at least 25%, at least 50%, at least 70%, at least 80%, at least 90%, at least 95%, or at least 99% by weight of the above-described hydrofluoroolefins based on the total weight of the working fluid. In addition to the hydrofluoroolefins, the working fluids may include a total of up to 75%, up to 50%, up to 30%, up to 20%, up to 10%, up to 5%, or up to 1% by weight of one or more of the following components: alcohols, ethers, alkanes, alkenes, perfluorocarbons, perfluorinated tertiary amines, perfluoroethers, cycloalkanes, esters, ketones, oxiranes, aromatics, siloxanes, hydrochlorocarbons, hydrochlorofluorocarbons, hydrofluorocarbons, hydrofluoroolefins, hydrochlorofluoroolefins, hydrofluoroethers, or mixtures thereof, based on the total weight of the working fluid. Such additional components can be chosen to modify or enhance the properties of a composition for a particular use. Minor amounts of optional components can also be added to the working fluids to impart particular desired properties for particular uses. Useful components can include conventional additives such as, for example, surfactants, coloring agents, stabilizers, anti-oxidants, flame retardants, and the like, and mixtures thereof.

The hydrofluoroolefins of the present disclosure (or a normally liquid working fluid comprising, consisting, or consisting essentially thereof) can be used in various applications. For example, the hydrofluoroolefins are believed to possess the required stability as well as the necessary short atmospheric lifetime and hence low global warming potential to make them viable environmentally-friendly candidates for high temperature heat transfer applications.

In some embodiments, the present disclosure is further directed to an apparatus for heat transfer that includes a device and a mechanism for transferring heat to or from the device. The mechanism for transferring heat may include a heat transfer working fluid that includes a hydrofluoroolefin of the present disclosure.

The provided apparatus for heat transfer may include a device. The device may be a component, work-piece, assembly, etc. to be cooled, heated or maintained at a predetermined temperature or temperature range. Such devices include electrical components, mechanical components and optical components. Examples of devices of the present disclosure include, but are not limited to microprocessors, wafers used to manufacture semiconductor devices, power control semiconductors, electrical distribution switch gear, power transformers, circuit boards, multi-chip modules, packaged and unpackaged semiconductor devices, lasers, chemical reactors, fuel cells, and electrochemical cells. In some embodiments, the device can include a chiller, a heater, or a combination thereof.

In yet other embodiments, the devices can include electronic devices, such as processors, including microprocessors. As these electronic devices become more powerful, the amount of heat generated per unit time increases. Therefore, the mechanism of heat transfer plays an important role in processor performance. The heat-transfer fluid typically has good heat transfer performance, good electrical compatibility (even if used in “indirect contact” applications such as those employing cold plates), as well as low toxicity, low (or non-) flammability and low environmental impact. Good electrical compatibility suggests that the heat-transfer fluid candidate exhibit high dielectric strength, high volume resistivity, and poor solvency for polar materials. Additionally, the heat-transfer fluid should exhibit good mechanical compatibility, that is, it should not affect typical materials of construction in an adverse manner.

The provided apparatus may include a mechanism for transferring heat. The mechanism may include a heat transfer fluid. The heat transfer fluid may include one or more hydrofluoro olefins of the present disclosure. Heat may be transferred by placing the heat transfer mechanism in thermal contact with the device. The heat transfer mechanism, when placed in thermal contact with the device, removes heat from the device or provides heat to the device, or maintains the device at a selected temperature or temperature range.

The direction of heat flow (from device or to device) is determined by the relative temperature difference between the device and the heat transfer mechanism.

The heat transfer mechanism may include facilities for managing the heat-transfer fluid, including, but not limited to pumps, valves, fluid containment systems, pressure control systems, condensers, heat exchangers, heat sources, heat sinks, refrigeration systems, active temperature control systems, and passive temperature control systems.

Examples of suitable heat transfer mechanisms include, but are not limited to, temperature controlled wafer chucks in plasma enhanced chemical vapor deposition (PECVD) tools, temperature-controlled test heads for die performance testing, temperature-controlled work zones within semiconductor process equipment, thermal shock test bath liquid reservoirs, and constant temperature baths. In some systems, such as etchers, ashers, PECVD chambers, vapor phase soldering devices, and thermal shock testers, the upper desired operating temperature may be as high as 170° C., as high as 200° C., or even as high as 240° C.

Heat can be transferred by placing the heat transfer mechanism in thermal contact with the device. The heat transfer mechanism, when placed in thermal contact with the device, may remove heat from the device or provide heat to the device, or maintain the device at a selected temperature or temperature range. The direction of heat flow (from device or to device) is determined by the relative temperature difference between the device and the heat transfer mechanism. The provided apparatus can also include refrigeration systems, cooling systems, testing equipment and machining equipment. In some embodiments, the provided apparatus can be a constant temperature bath or a thermal shock test bath. In some systems, such as etchers, ashers, PECVD chambers, vapor phase soldering devices, and thermal shock testers, the upper desired operating temperature may be as high as 170° C., as high as 200° C., or even higher.

In some embodiments, the hydrofluoroether olefins of the present disclosure may be used as a heat transfer agent for use in vapor phase soldering. In using the compounds of the present disclosure in vapor phase soldering, the process described in, for example, U.S. Pat. No. 5,104,034 (Hansen) can be used, which description is hereby incorporated by reference in its entirety. Briefly, such process includes immersing a component to be soldered in a body of vapor comprising at least one hydrofluoro olefin of the present disclosure to melt the solder. In carrying out such a process, a liquid pool of hydrofluoro olefin (or working fluid that includes the hydrofluoro olefin) is heated to boiling in a tank to form a saturated vapor in the space between the boiling liquid and a condensing means.

A workpiece to be soldered is immersed in the vapor (at a temperature of greater than 170° C., greater than 200° C., greater than 230° C., or even greater), whereby the vapor is condensed on the surface of the workpiece so as to melt and reflow the solder. Finally, the soldered workpiece is then removed from the space containing the vapor.

Objects and advantages of this disclosure are further illustrated by the following examples, but the particular materials and amounts thereof recited in these examples, as well as other conditions and details, should not be construed to unduly limit this disclosure.

### EXAMPLES

Compositions of the present disclosure were prepared using materials outlined in Table 1 below.

TABLE 1

| Chemical | Description | Source |
|---|---|---|
| 1,1,1,2,3,4,5,5,5- | HFP Dimer 2 isomers: | 3M Foam Additive FA- |
| nonafluoro-4- | | 188, 3M, St. Paul, MN. |
| trifluoromethyl-pent-2-ene | 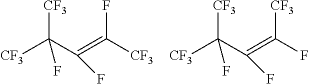 [SMILES: F/C(=C(/F)C(F)(C(F)(F)F)C(F)(F)F)C(F)(F)F ; F/C(=C(\F)C(F)(C(F)(F)F)C(F)(F)F)C(F)(F)F] | |
| N,N-Dimethylformamide | (CH₃)₂NC(O)H | EMD Chemicals, Inc. |
| | | Gibbstown, NJ |
| Potassium Fluoride | KF | Sigma Aldrich, |
| | | Milwaukee, WI |
| trans-1,4-dibromo-2- | Trans BrCH₂CH═CHCH₂Br | AK Scientific Inc., Union |
| butene | | City, CA |
| Potassium Iodide | KI | Sigma Aldrich, |
| | | Milwaukee, WI |
| cis-1,4-dichloro-2-butene | Cis ClCH₂CH═CHCH₂Cl | VWR International, LLC, |
| | | Radnor, PA |
| Methyltrialkyl(C₈-C₁₀) | CH₃(C₈H₁₇)₃N⁺Cl⁻ | Sigma Aldrich, |
| ammonium chloride | | Milwaukee, WI |

### Example 1 (Ex 1)—Synthesis of trans-CF3CF2CF2C(CF3)2-CH2CH═CHCH2-C(CF3)2CF2CF2CF3

A 600 ml stainless steel reactor was fitted with a mixer and charged with 185 g N,N-dimethylformamide, 34 g methyltrialkyl(C8-C10) ammonium chloride, 43 g potassium fluoride, 185 g 1,1,1,2,3,4,5,5,5-nonafluoro-4-trifluoromethyl-pent-2-ene, and 60 g trans-1,4-dibromo-2-butene. The reactor was heated to 40° C., with stirring (500 rpm), and allowed to react at this temperature for 72 hours. At the end of reaction, the reactor contents were vacuum distilled at 20 torr and 150° C. The distillate was condensed by dry ice and collected in a flask. 180 g FC phase in the distillate was collected. The FC phase was then washed by 180 g water and allowed to phase split. 161 g bottom phase was collected. Analysis of the bottom phase by Gas Chromatography indicated 89% purity of trans-CF3CF2CF2C(CF3)2-CH2CH═CHCH2-C(CF3)2CF2CF2CF3. This material was then further purified by vacuum fractionation to yield a 99% pure fluid.

### Example 2 (Ex 2)—Synthesis of cis-CF3CF2CF2C(CF3)2-CH2CH═CHCH2-C(CF3)2CF2CF2CF3

A 600 ml stainless steel reactor fitted with mixer and charged with 200 g N,N-dimethylformamide, 49 g methyltrialkyl(C8-C10) ammonium chloride, 60 g potassium fluoride, 4 g potassium iodide, 260 g 1,1,1,2,3,4,5,5,5-nonafluoro-4-trifluoromethyl-pent-2-ene, and 47 g trans-1,4-dichloro-2-butene were added. The reactor was heated to 40° C., with stirring (500 rpm), and allowed to react at this temperature for 48 hours. At the end of reaction, the reactor contents were vacuum distilled at 20 torr and 150° C. The distillate was condensed by dry ice and collected in a flask. 270 g FC phase in the distillate was collected. The FC phase was then washed by 250 g water and allowed to phase split. 245 g bottom phase was collected. Analysis of the bottom phase by Gas Chromatography indicated 91% purity of cis-CF3CF2CF2C(CF3)2-CH2CH═CHCH2-C(CF3)2CF2CF2CF3. This material was then further purified by vacuum fractionation to yield a 99% pure fluid.

### Material Characterization

Compositions of the present disclosure as well as a comparative examples (CE 1-GALDEN PFPE HS-240 from Solvay, Cranbury, N.J.; CE 2—GALDEN PFPE HT-270 from Solvay, Cranbury, N.J.; CE 3—FLUORINERT FC-43 from 3M Company, St Paul, Minn.) were characterized for a number of thermophysical properties. (Some of the properties for GALDEN PFPE HS-240 were obtained from data published by Solvay, Cranbury, N.J.)

The dielectric breakdown strengths of Example 1 and 2 were determined according to ASTM D877, using a model LD60 liquid dielectric test set available from Phenix Technologies, Accident, MD. The breakdown strengths for Example 1 and 2 were both 50 kV/m.

Kinematic Viscosity was measured using a Schott AVS 350 Viscosity Timer, Analytical Instrument No. 341. For temperatures below 0° C., a Lawler temperature control bath, Analytical Instrument No. 320, was used. The viscometers used for all temperature are 545-10 and 23. Viscometers were also corrected using the Hagenbach correction.

Vapor Pressure was measured using the stirred-flask ebuilliometer method described in ASTM E-1719-97 “Vapor Pressure Measurement by Ebuilliometry”. This method is also referred to as “Dynamic Reflux”. Boiling Point was measured using ASTM D1120-94 “Standard Test Method for Boiling Point of Engine Coolants.

Pour Point was measured by placing a sealed glass vial containing 3 mL of the fluid into a refrigerated bath, adjusting temperature incrementally and checking for pouring. Pouring is defined as visible movement of the material during a five second count. This criterion is specified in ASTM D97.

Density was measured using an Anton Paar DMA5000M Density Meter, Analytical Instrument No. 1223.

Specific heat capacity was measured using conventional modulated differential scanning calorimetry (MDSC).

Heat of vaporization was calculated from the vapor pressure vs. temperature curve using the Clausius-Clapeyron Equation.

Table 2 shows some thermophysical properties of exemplary hydrofluoroolefins and a comparative material (CE 1)

TABLE 2

| | | Normal | | | Heat of | Specific | Vapor | Dielectric | |
|---|---|---|---|---|---|---|---|---|---|
| | | Boiling | Pour | Viscosity | Vaporization | Heat | Pressure | Strength | Density |
| | | Point | Point | @ 25° C. | @ BPT | Capacity | @ 25° C. | @2.54 gap | @ 25° C. |
| Ex | Material | (° C.) | (° C.) | (×10⁻³m²/s) | (kJ/kg) | (J/kg · K) | (ton) | (kV) | (kg/m³) |
| CE1 | GALDEN PFPE | 240 | n/a | 5.3 | 63 | 973 | 1 | 40 | 1.82 |
| | HS-240 | | | | | | | | |
| Ex 1 | trans- | 233 | −52 | 11.4 | 77 | 1110 | 0.007 | 50 | 1.78 |
| | C [symbol] F₁₃C₄H₆C₆F [symbol] | | | | | | | | |
| Ex 2 | cis-C [symbol] F₁₃C₄H₆C₆F [symbol] | 223 | −57 | 6.5 | 75 | 1050 | 0.003 | 50 | 1.78 |

[symbol]

indicates data missing or illegible when filed

Thermal stability was measured by placing a sealed monel bomb containing 10 g fluid in an oven that was controlled at testing temperature (e.g., 150° C. or 223° C.) for 7 days. At the end of the 7-day testing period, the bomb was cooled to room temperature, opened, and the fluid was poured out for fluoride ion analysis. The fluid sample was analyzed using a fluoride meter (ORION EA 940 meter/F-ISE). The fluorochemical sample was extracted using ultra pure DI water. One milliliter of extracted sample was buffered 1:1 with TISAB II. The fluoride meter was calibrated using a series of 1, 2, 10 and 100 ppm F as sodium fluoride solution (ORION). The results of the thermal stability test are shown in Table 3. It should be noted that Ex 1 (trans-C6F13C4H6C6F13) was deaerated using a vacuum for several minutes prior to testing.

TABLE 3

| | | Testing | | Fluoride |
|---|---|---|---|---|
| | | Temperature | Testing Time | Generated |
| Ex | Material | (° C.) | (hours) | (ppm) |
| CE 2 | GALDEN PFPE HT-270 | 233 | 168 | 0.30 |
| Ex 1 | trans-C₆F₁₃C₄H₆C₆F₁₃ | 233 | 168 | 0.25 |
| CE 3 | FLUORINERT FC-43 | 150 | 168 | 0.04 |
| Ex 2 | cis-C₆F₁₃C₄H₆C₆F₁₃ | 150 | 168 | 2.0 |

The material Ex 1 was also tested for its stability with lead-free solder flux under its normal boiling temperature and atmospheric conditions. 14 g of the testing fluid, along with 0.44 g of Alpha OM-340 solder paste (available from Alpha, Altoona Pa.), was added to a 25 ml glass flask fitted with an overhead water condenser and a dry ice trap. The flask was then heated at 233° C. to keep it boiling and refluxing for a period of 5 days. Analysis of the resulting fluid by Gas chromatography (GC) indicated that the change of the fluid purity was less than 0.01%. This result is indicative that there was no reaction between the trans-C6F13C4H6C6F13 (Ex 1) and the solder flux which is typically used for vapor phase soldering.

Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows. All references cited in this disclosure are herein incorporated by reference in their entirety.

1. A composition comprising a hydrofluoroolefin represented by the following general formula (I):
Rf—CH2CH═CHCH2-Rf (I)
wherein Rf is a perfluoroalkyl group having 6 carbon atoms; and
wherein the hydrofluoroolefin is a liquid at room temperature.

2. A composition according to claim 1, wherein the hydrofluoroolefin is represented by the following formula:
CF3CF2CF2C(CF3)2CH2CH═CHCH2C(CF3)2CF2CF2CF3.

3. A composition according to claim 1, wherein the hydrofluoroolefin comprises the cis isomer.

4. A composition according to claim 1, wherein the hydrofluoroolefin comprises the trans isomer.

5. A working fluid comprising a composition according to claim 1, wherein the hydrofluoroolefin is present in the working fluid at an amount of at least 50% by weight based on the total weight of the working fluid.

6. An apparatus for heat transfer comprising:
a device; and
a mechanism for transferring heat to or from the device, the mechanism comprising a heat transfer fluid that comprises a composition or working fluid according to any one of the previous claims.

7. An apparatus for heat transfer according to claim 6, wherein the device is selected from a microprocessor, a semiconductor wafer used to manufacture a semiconductor device, a power control semiconductor, an electrochemical cell, an electrical distribution switch gear, a power transformer, a circuit board, a multi-chip module, a packaged or unpackaged semiconductor device, a fuel cell, and a laser.

8. An apparatus according to claim 6, wherein the mechanism for transferring heat is a component in a system for maintaining a temperature or temperature range of an electronic device.

9. An apparatus according to claim 6, wherein the device comprises an electronic component to be soldered.

10. An apparatus according to claim 6, wherein the mechanism comprises vapor phase soldering.

11. A method of transferring heat comprising:
providing a device; and
transferring heat to or from the device using a heat transfer fluid that comprises a composition or working fluid according to claim 1.
